(19) United States
(12) Patent Application Publication
(10) Pub. No.: US 2019/0179982 A1
(43) Pub. Date: Jun. 13, 2019

(54) UTILIZING A SUBSCRIBED PLATFORM AND CLOUD-COMPUTING TO MODEL DISEASE RISK IN AGRONOMIC CROPS FOR MANAGEMENT DECISIONS

(71) Applicant: AgReliant Genetics, LLC, Westfield, IN (US)

(72) Inventors: Brittiney N. Reese, Thorntown, IN (US); Noah D. Freeman, Frankfort, IN (US)

(21) Appl. No.: 16/217,890

(22) Filed: Dec. 12, 2018

Related U.S. Application Data
(60) Provisional application No. 62/598,301, filed on Dec. 13, 2017.

(51) Int. Cl. G06F 17/50; G01N 33/00; A01G 7/00

(52) U.S. Cl. CPC G06F 17/5009; H04L 67/10; A01G 7/00; G01N 33/0098

(57) ABSTRACT

Technologies for assessing a disease risk include receiving a geographical area of a geospatial unit defined by a user of the computing device, receiving input variables associated with the geospatial unit from one or more input sources of the computing device, determining a disease risk by performing a modeled disease risk assessment as a function of the input variables and a set of predefined variables, generating a visual representation that illustrates the disease risk of plants at the geospatial unit based on the modeled disease risk assessment, outputting the visual representation on the computing device, and transmitting, in response to determining that the disease risk exceeds a predefined threshold, a notification to a user of the computing device indicating that the geospatial unit is at risk.

This application claims priority under 35 U.S.C. § 119 to U.S. Patent Application Ser. No. 62/598,301, which was filed on Dec. 13, 2017 and is expressly incorporated herein by reference.

### TECHNICAL FIELD

The present disclosure relates generally to agricultural technology, and more particularly, to the detection, monitoring, and control of disease risk of agronomic crops.

### BACKGROUND

World population is estimated to reach 9.8 billion by 2050. With increasing population, agriculture is challenged to increase efficiency and yields to meet a global demand of commodities. In 2016, 10.8% of total corn bushels were estimated to have been lost from corn pathogens in the US and Canada. Although corn is a host to many pathogens in Northern America, three classes of disease (foliar, stalk rots, and ear rots) can be detrimental to yields and grain quality. In 2016, 235 million bushels were estimated to be lost from Gray Leaf Spot, 197.8 million bushels to Anthracnose Stalk Rot & Top Die Back, and 173.2 million bushels to Diploid Ear Rot.

### SUMMARY

According to one aspect of the disclosure, a computing system is disclosed. The computing system device includes a computing device and a server computing device, which may be communicatively coupled to the computing device. The server computing device may be configured to receive a geographical area of a geospatial unit defined by a user of the computing device, receive input variables associated with the geospatial unit from one or more input sources of the computing device, determine the disease risk by performing a modeled disease risk assessment as a function of the input variables and a set of predefined variables, generate a visual representation that illustrates the disease risk of plants at the geospatial unit based on the modeled disease risk assessment, output the visual representation on the computing device, and transmit, in response to determining that the disease risk exceeds a predefined threshold, a notification to a user of the computing device indicating that the geospatial unit is at risk. In some embodiments, the visual representation may include a visual timeline. In some embodiments, to generate the visual representation that illustrates the disease risk at the geospatial unit may include to generate a visual indication on the timeline at which the disease risk exceeds the predefined threshold. For example, the visual indication may represent specific time at which the plants in the geospatial unit are likely to develop a disease.

In some embodiments, to receive the input variables from one or more input sources may include to receive data from the user, one or more administrators, and/or one or more third-parties. In some embodiments, to receive input variables from one or more input sources may include to receive platform generated data, remote tracking and input data, and/or data from platform databases, external databases, and/or application programming interface. For example, the input variables may include hybrid/variety genetics, hybrid/variety disease ratings, hybrid/variety seed treatments, weather forecasts, current weather and past weather data, crop rotation history, tillage, irrigation, and other cultural practices, topographic actual and generated data, soil test and generated data, yield data, imagery actual and generated data, disease history, planting date, fertility plans, fertility history, crop growth stage (actual and/or calculated), crop protectant history, scouting data, and/or other forms of disease observation and tracking. Additionally, in some embodiments, the input variables may include crop rotation history of the geospatial unit indicative of a previous crop assigned in the geospatial unit. The crop rotation history may be automatically determined by analyzing satellite images of the geospatial unit.

In some embodiments, to determine the disease risk may include to analyze the input variables to determine current, short-term, and/or long-term disease risk levels for individual plant pathogens in the geospatial unit. Additionally, in some embodiments, to determine a disease risk may include to perform a modeled disease risk assessment on both the geospatial unit and sub-units within the geospatial unit based on sub-unit variables that vary across the defined geospatial unit. In some embodiments, the visual representation may include information regarding a pathogen related to the disease, information regarding the disease, scouting information, general management and cultural management practices, and/or pesticide specific management practices. In some embodiments, the notification may include information regarding a pathogen related to the disease, information regarding the disease, scouting information, general management and cultural management practices, and/or pesticide specific management practices.

In some embodiments, the server computing device may be further configured to receive a plurality of geospatial units and simultaneously determine a disease risk at each geospatial unit by performing a modeled disease risk assessment as a function of the input variables and a set of predefined variables for the each geospatial unit. In some embodiments, to determine the disease risk may include to determine a hybrid resistance level of the plants at the geospatial unit, and to generate the visual representation may include to change the visual representation based on the hybrid resistance level. Additionally, to determine the disease risk may include to generate a low risk visual representation on the computing device in response to determining that the hybrid resistance level is above a predefined level.

In some embodiments, to determine the disease risk may include to determine whether secondary input variables of the geospatial unit satisfy a predefined disease condition in response to determining that the hybrid resistance level is within a predefined range. For example, the secondary input variables may include temperature, relative humidity, and chance of precipitation. In some embodiments, to determine the disease risk may include to determine, in response to determining that the secondary input variables satisfy the predefined disease condition, crop rotation history and tillage practices of the geospatial unit to determine the disease risk and determine whether the disease risk exceeds the predefined threshold.

In some embodiments, to generate the visual representation on the computing device may include to generate, in response to determining that the disease risk exceeds the predefined threshold, the visual representation indicative of the disease risk at the geospatial unit that includes information regarding a pathogen related to the disease, information regarding the disease, scouting information, general management and cultural management practices, and/or pesticide specific management practices.

According to another aspect, a method for assessing a disease risk includes receiving a geographical area of a geospatial unit defined by a user of the computing device, receiving input variables associated with the geospatial unit from one or more input sources of the computing device, determining the disease risk by performing a modeled disease risk assessment as a function of the input variables and a set of predefined variables, generating a visual representation that illustrates the disease risk of plants at the geospatial unit based on the modeled disease risk assessment, outputting the visual representation on the computing device, and transmitting, in response to determining that the disease risk exceeds a predefined threshold, a notification to a user of the computing device indicating that the geospatial unit is at risk. In some embodiments, the visual representation may include a visual timeline. In some embodiments, generating the visual representation that illustrates the disease risk at the geospatial unit may include generating a visual indication on the timeline at which the disease risk exceeds the predefined threshold. In some embodiments, the visual indication may represent specific time at which the plants in the geospatial unit are likely to develop a disease.

Further, in some embodiments, receiving the input variables from one or more input sources may include receiving data from the user, one or more administrators, and/or one or more third-parties. In some embodiments, receiving input variables from one or more input sources may include receiving platform generated data, remote tracking and input data, and/or data from platform databases, external databases, and/or application programming interface. For example, the input variables may include hybrid/variety genetics, hybrid/variety disease ratings, hybrid/variety seed treatments, weather forecasts, current weather and past weather data, crop rotation history, tillage, irrigation, and other cultural practices, topographic actual and generated data, soil test and generated data, yield data, imagery actual and generated data, disease history, planting date, fertility plans, fertility history, crop growth stage (actual and/or calculated), crop protectant history, scouting data, and/or other forms of disease observation and tracking. In some embodiments, the input variables may include crop rotation history of the geospatial unit indicative of a previous crop assigned in the geospatial unit. The crop rotation history may be automatically determined by analyzing satellite images of the geospatial unit.

In some embodiments, determining the disease risk may include analyzing the input variables to determine current, short-term, and/or long-term disease risk for individual plant pathogens in the geospatial unit. In some embodiments, determining the disease risk by performing a modeled disease risk assessment may include determining a current disease risk of the plants at the geospatial unit. Additionally, generating the visual representation may include generating a timeline that includes a visual indication at which the current disease risk exceeds a corresponding predefined threshold and transmitting the notification to the user may include transmitting, in response to determining that the current disease risk exceeds the corresponding predefined threshold, a notification to a user of the computing device indicating that the geospatial unit is at risk.

In some embodiments, determining the disease risk by performing a modeled disease risk assessment may include determining a short-term disease risk of the plants at the geospatial unit and generating the visual representation may include generating a timeline that includes a visual indication at which the short-term disease risk exceeds a corresponding predefined threshold. Additionally, transmitting the notification to the user may include transmitting, in response to determining that the short-term disease risk exceeds the corresponding predefined threshold, a notification to a user of the computing device indicating that the geospatial unit is at risk.

In some embodiments, determining the disease risk by performing a modeled disease risk assessment may include determining current and short-term disease risks of the plants at the geospatial unit and generating the visual representation may include generating a timeline that includes visual indications at which each of the current and short-term disease risks exceeds a corresponding predefined threshold. Additionally, transmitting the notification to the user may include transmitting, in response to determining that each of the current and short-term disease risks exceed the corresponding predefined threshold, a notification to a user of the computing device indicating that the geospatial unit is at risk.

In some embodiments, determining the disease risk may include performing a modeled disease risk assessment on both the geospatial unit and sub-units within the geospatial unit based on sub-unit variables that vary across the defined geospatial unit. In some embodiments, the visual representation may include information regarding a pathogen related to the disease, information regarding the disease, scouting information, general management and cultural management practices, and/or pesticide specific management practices. Additionally, in some embodiments, the notification may include information regarding a pathogen related to the disease, information regarding the disease, scouting information, general management and cultural management practices, and/or pesticide specific management practices.

In some embodiments, the method may further include receiving a plurality of geospatial units and simultaneously determine a disease risk at each geospatial unit by performing a modeled disease risk assessment as a function of the input variables and a set of predefined variables for the each geospatial unit.

In some embodiments, determining the disease risk may include determining a hybrid resistance level of the plants at the geospatial unit, and generating the visual representation may include changing the visual representation based on the hybrid resistance level. In some embodiments, determining the disease risk may include generating a low risk visual representation on the computing device in response to determining that the hybrid resistance level is above a predefined level. Further, determining the disease risk may include determining whether secondary input variables of the geospatial unit satisfy a predefined disease condition in response to determining that the hybrid resistance level is within a predefined range. For example, the secondary input variables may include temperature, relative humidity, and chance of precipitation. Further, determining the disease risk may include determining, in response to determining that the secondary input variables satisfy the predefined disease condition, crop rotation history and tillage practices of the geospatial unit to determine the disease risk and determine whether the disease risk exceeds the predefined threshold.

In some embodiments, generating the visual representation on the computing device may include generating, in response to determining that the disease risk exceeds the predefined threshold, the visual representation indicative of the disease risk at the geospatial unit that includes information regarding a pathogen related to the disease, information regarding the disease, scouting information, general management and cultural management practices, and/or pesticide specific management practices.

### BRIEF DESCRIPTION OF THE DRAWINGS

The detailed description particularly refers to the following figures, in which:

FIG. 1 is a simplified flow diagram of at least one embodiment of a method for a disease risk assessment modeling that may be performed by a computing system;

FIG. 2 is a diagrammatic view of at least one embodiment of exemplary input sources and exemplary input variables for a disease risk assessment modeling;

FIG. 3 is a diagrammatic view of at least one embodiment of exemplary output variables generated based on input variables via cloud-computing using one or more disease model;

FIGS. 4A-4C are simplified illustrations of at least one embodiment of geospatial units created in a platform on a user's computing device with assigned crop and crop rotation;

FIGS. 5-7 are simplified illustrations of at least one embodiment of a Seed Plan platform;

FIGS. 8-10 are simplified illustrations of at least one embodiment a Field Plan platform;

FIG. 11 is a simplified illustration of at least one embodiment of a Fertility Plan platform;

FIG. 12 is a simplified illustration of at least one embodiment of a Soil Data platform;

FIG. 13 is a simplified illustration of at least one embodiment of a Scouting Data platform;

FIG. 14 is a simplified illustration of at least one embodiment of creating new Field Records as input variables;

FIG. 15 is a simplified illustration of at least one embodiment of a home screen of the platform displayed on the computing device illustrating a disease risk alert for a current risk event next to the affected geospatial unit;

FIG. 16 is a simplified illustration of at least one embodiment of a visual representation on the computing device indicating a disease insight alert on a field timeline for a current risk event;

FIG. 17 is a simplified illustration of at least one embodiment of a visual representation on the computing device generating disease insight outputs on a field screen for a current risk event on the computing device;

FIG. 18 is a simplified illustration of at least one embodiment of a visual representation on the computing device indicating sections of disease risk output information to grower a current risk event;

FIG. 19 is a simplified illustration of at least one embodiment of a home screen of the platform displayed on the computing device illustrating a disease insight alert for a short-term risk event next to the effected geospatial unit;

FIG. 20 is a simplified illustration of at least one embodiment of a visual representation on the computing device indicating a disease insight alert on a field timeline for a short-term risk event;

FIG. 21 is a simplified illustration of at least one embodiment of a visual representation on the computing device generating disease insight outputs on a field screen for a short-term risk event;

FIG. 22 is a simplified illustration of at least one embodiment of a visual representation on the computing device indicating sections of disease risk output information to grower a short-term risk event; and

FIG. 23 is a simplified illustration of at least one embodiment of a home screen of the platform displayed on the computing device illustrating a disease insight alert for a long-term forecast disease risk next to the effected geospatial unit.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Advancing technologies have driven agriculture to become a complex system of inputs, outputs, and management decisions. Although growers are bombarded with a plethora of information and data, the growers are challenged with translating these variables into economically valuable management decisions as there are multiple parameters and environmental variables that can influence disease development within a growing season.

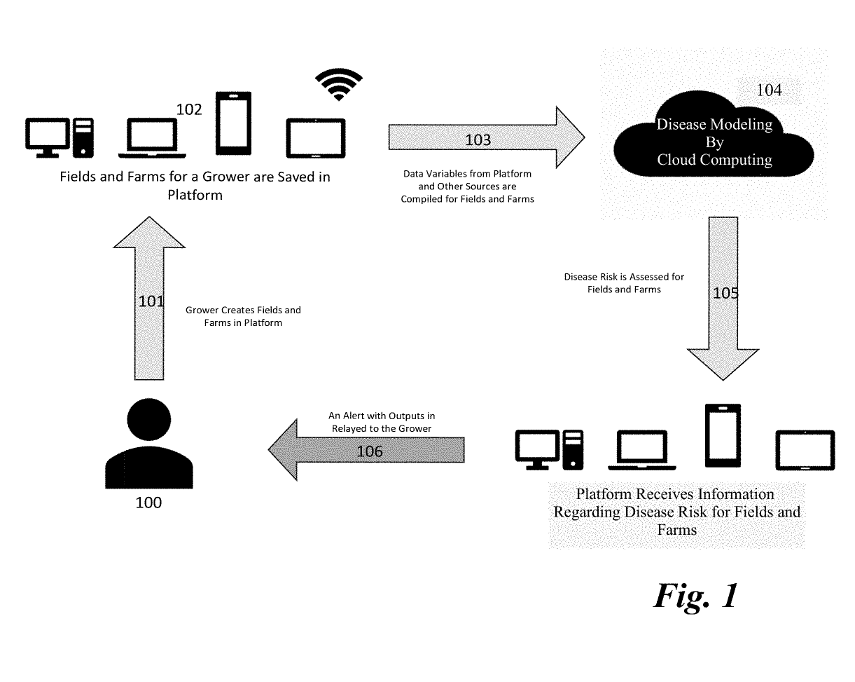

Referring to FIG. 1, a computing device (102) of a user or a subscriber (100) is communicatively coupled to a server computing device. The user (100) is subscribed to a platform executed on the server computing device in a cloud-based computing environment on a computing device (102). The computing device (102) may be embodied as a phone, a laptop computer, a desktop computer, a tablet computing device, or other computing device capable of a wireless connection. The user (100) inputs information regarding farms and fields (101) into the platform on the computing device (102). The user defined a geographical area of each of the farms and fields as a geospatial unit. The computing device (102) transmits information to a processing module (103) of the server computing device for disease modeling (104). Disease models and risks (105) are relayed back to the computing device (102). An alert with detailed output (106) is presented on the computing device (102) for the subscriber to make informed management decisions. In other words, the server computing device is configured to provide disease risk modeling for a geospatial unit defined by the user by allowing the integration of various sourced input variables for evaluating disease risk and alerting a subscriber to the risk at the defined geospatial unit.

The server computing device may be embodied as any type of computing device capable of executing the platform to perform the functions described herein including, but not limited to, a server, a desktop computer, a laptop computer, a tablet computing device, and/or any other type of computing device. The illustrative server computing device includes a processor, a memory, and data storage. It should be appreciated that the server computing device may include other or additional components, such as those commonly found in computing devices (e.g., input/output subsystems, communication circuitry, peripheral devices, displays, etc.) in other embodiments. Additionally, in some embodiments, one or more of the illustrative components may be incorporated in, or otherwise from a portion of, another component.

A paradigm for disease development includes three elements: Host, Environment, and Pathogen. Each of the elements may be further broken down into a suite of input variables contributing to the overall risk of disease development. In the illustrative embodiment, the input variables may be manually entered by a grower or a subscriber (204). Manual entry of grower (204) includes any data entered into the platform by the subscriber. For example, manual entry of field records, field data, geospatial data, weather data, and any other disease risk variables may be inputted by the grower. Additionally, in some embodiments, manual entry of input variables may be received from administrators (205). The administrators are those who work in the subscribers account in lieu of the subscriber. Manual entry of input variables may be also received from a third-party (206). The third party is one or more individuals or entities that collaborate or share a defined geospatial unit within the platform. Such individuals may include farm managers, agronomists, dealers, and other collaborators or cooperators.

Moreover, the input variables may also be received via an application programming interface (API) (207). The API (207) may be the input source of disease risk variables, and API data includes, but not limited to, weather data and forecasts, and/or disease tracking which may be automatically collected into a database of contributing variables for analysis of disease risk. Disease risk variables may be obtained from internal databases (208) and external databases (209). Examples of database data include, but are not limited to, hybrid data, rating data, soil data, weather data, and other input variables for a disease risk assessment.

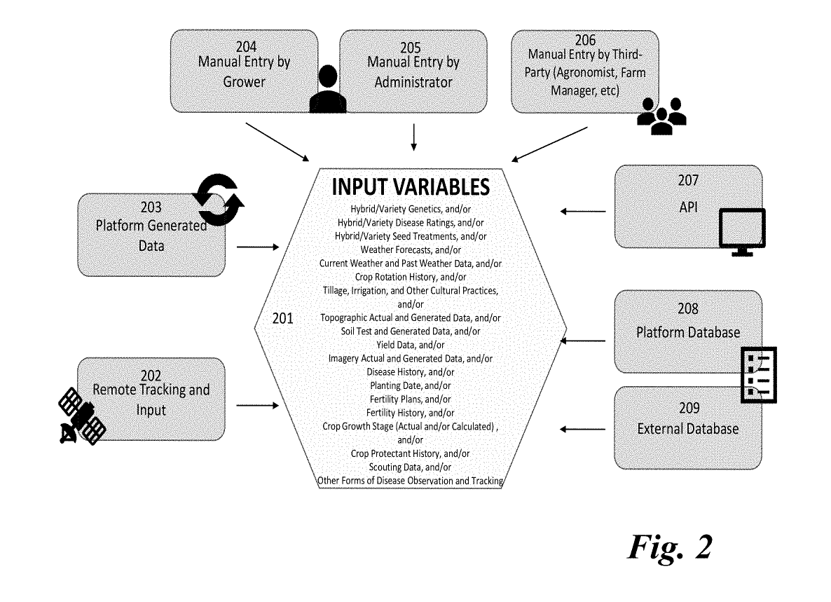

As shown in FIG. 2, exemplary input variables for disease risk assessment modeling (201) and exemplary input sources of the input variables may include Hybrid/Variety Genetics, Hybrid/Variety Disease Ratings, Hybrid/Variety Seed Treatments, Weather Forecasts, Current Weather and Past Weather Data, Crop Rotation History, Tillage, Irrigation, and Other Cultural Practices, Topographic Actual and Generated Data, Soil Test and Generated Data, Yield Data, Imagery Actual and Generated Data, Disease History, Planting Date, Fertility Plans, Fertility History, Crop Growth Stage (Actual and/or Calculated), Crop Protectant History, Scouting Data, and/or Other Forms of Disease Observation and Tracking. As discussed above, the input variables may be received for modeling through various inputs. For example, the input variables may be obtained from remote tracking and input data (202) and platform generated data (203). The remote tracking and input data (202) include data received from any third-party module, such as sensors or equipment data. The platform generated data (203) include any data extrapolated or computed from the platform, such as, but not limited to, functional soil maps and other internal models.

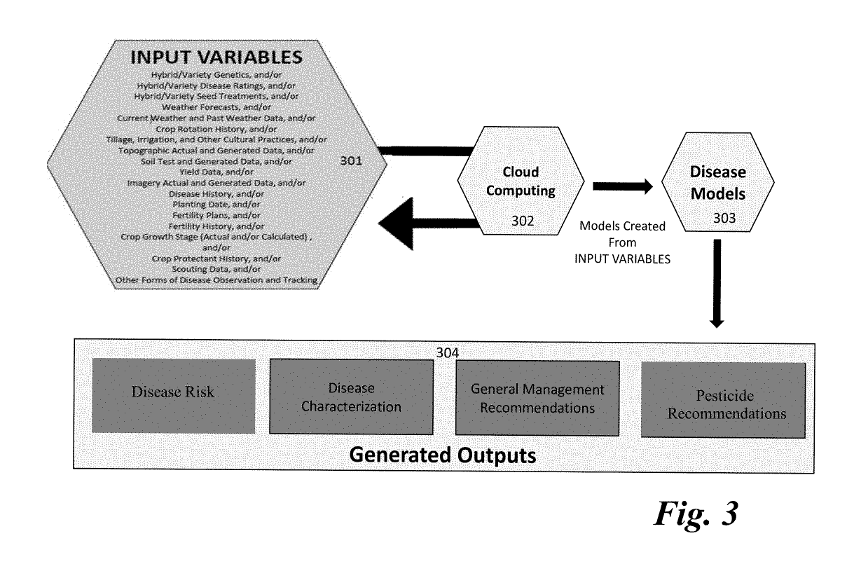

Referring now to FIG. 3, the input variables are compiled into the subscribed platform (301) executed on the server computing device. At continuous intervals, cloud-computing (302) is utilized to model the input variables for disease risks (303). When a threshold in the model is met, the server computing device generates outputs that may include disease risk, disease characterization, general management recommendations, and/or pesticide recommendations (304).

The platform is configured to model disease risk utilizing cloud-computing with the input variables. Algorithms with specific parameters is configured analyze disease risk for diseases at geospatial units defined by the subscriber. Modeling algorithms will run for diseases such as, but not exclusively, Northern Corn Leaf Blight, Southern Rust, Grey Leaf Spot, Gibberilla Ear Rot, Fusarium Ear Rot, Aspergillus Ear Rot, Diplodia Ear Rot, and individual Stalk Rots. In the illustrative embodiment, three levels of modeling will occur for each disease at each geospatial unit: current, short-term future, and long-term future risks. Algorithms for these levels assess risk through historic data, and/or current data, and/or short-term forecast data, and/or long-term forecast data, which is discussed in detail below.

The user will receive an alert in the platform on the computing device regarding geospatial units that are at risk of developing disease. Each alert is tailored to risk level and disease. The alert will provide insight into reason for risk, and/or general information regarding pathogen, and/or general information regarding disease, and/or scouting information, and/or general management and cultural management practices, and/or pesticide specific management practices.

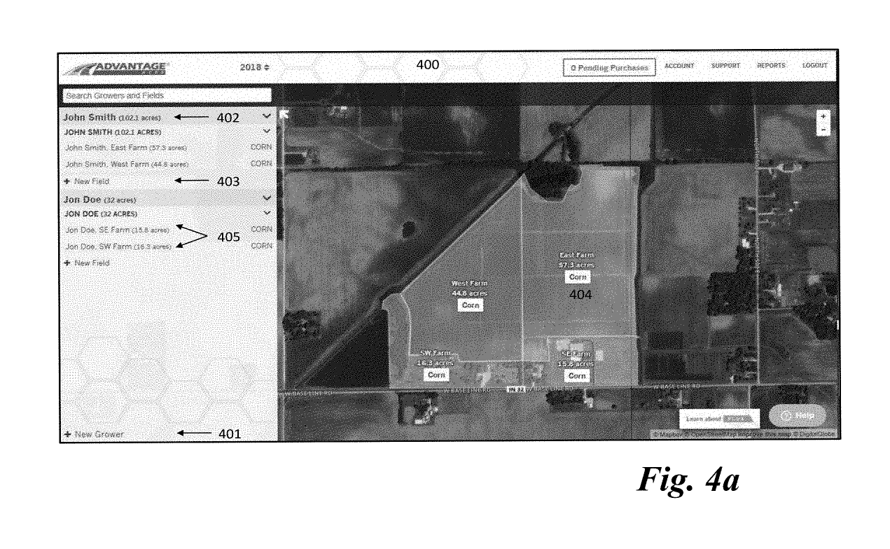

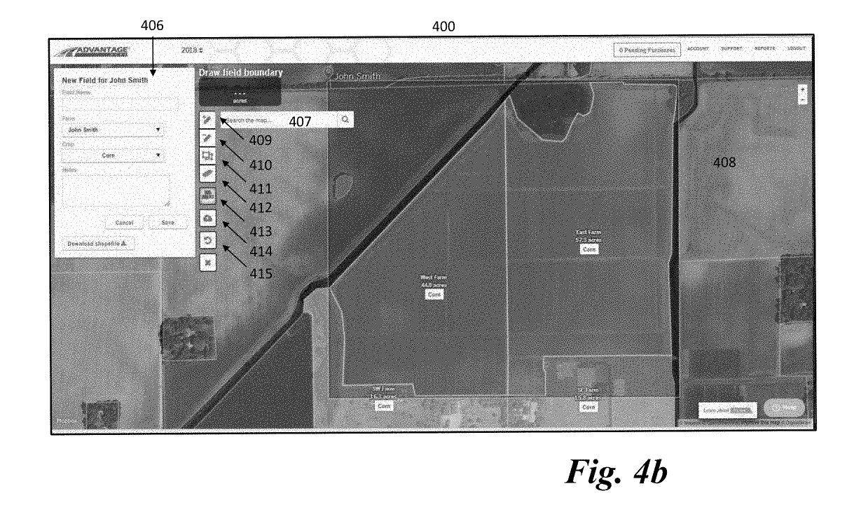

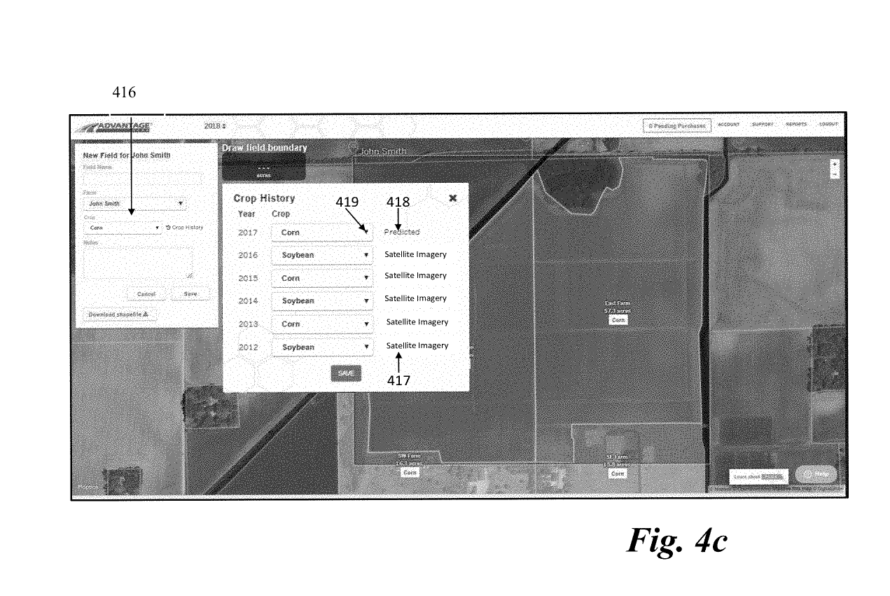

Referring to FIGS. 4A-4C, exemplary screen shots of the platform (400) displayed on the computing device are shown. As shown in FIG. 4A, the platform (400) may be accessed by a subscription on a computing device. In the platform (400), a new grower (401) is created. Once the grower is created, the name will appear in the list of growers (402) under the subscription. For created growers, a geospatial unit can be defined by creating a new field (403). The geospatial unit may be defined by selecting Common Land Units (CLU), free-drawing boundaries, or editing existing boundaries (404). The defined geospatial units are assigned to growers in the platform (405).

When creating defined geospatial units in the platform (400), the field can be named as a unique field and farm (406) as shown in FIG. 4B. The platform allows for the ability to search for geographic areas by name or address (407) on the platform satellite imagery map view screen (408). When the general geographic area of the desired unit is in the view screen (408), defined boundaries can be established. Boundaries can be free-drawn by the user by adding boundary lines (409) or by selecting CLUs (413). Overall boundaries can be edited by moving boundary lines (411), adding sections (409), and/or removing sections (410 and 411) of the defined geospatial unit. Shape files may also be uploaded to the platform (414). Any edits can be undone (415).

When the defined geospatial unit is created, previous crop may be assigned (416) as shown in FIG. 4C. Satellite imagery is used to assign previously planted crops (417). From crop rotation history, current crop is predicted (418). History and predictions can be manually overridden by the user (419).

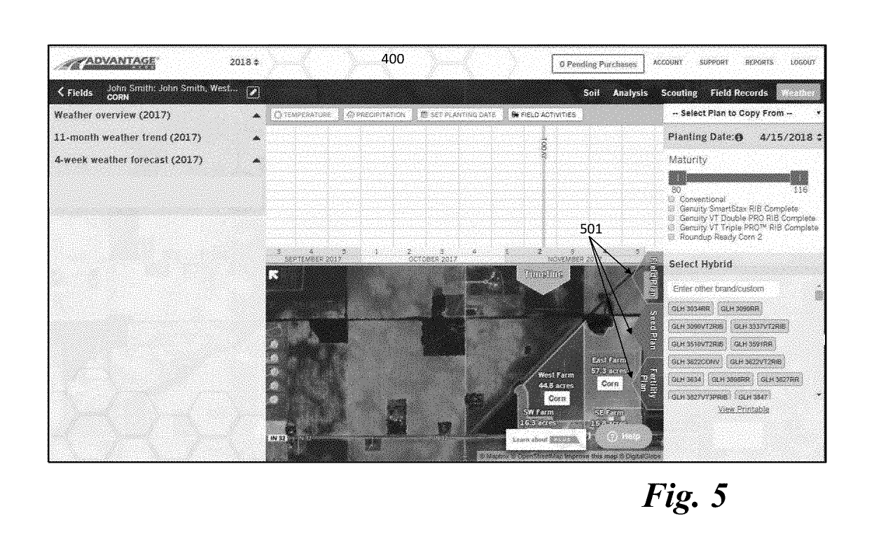

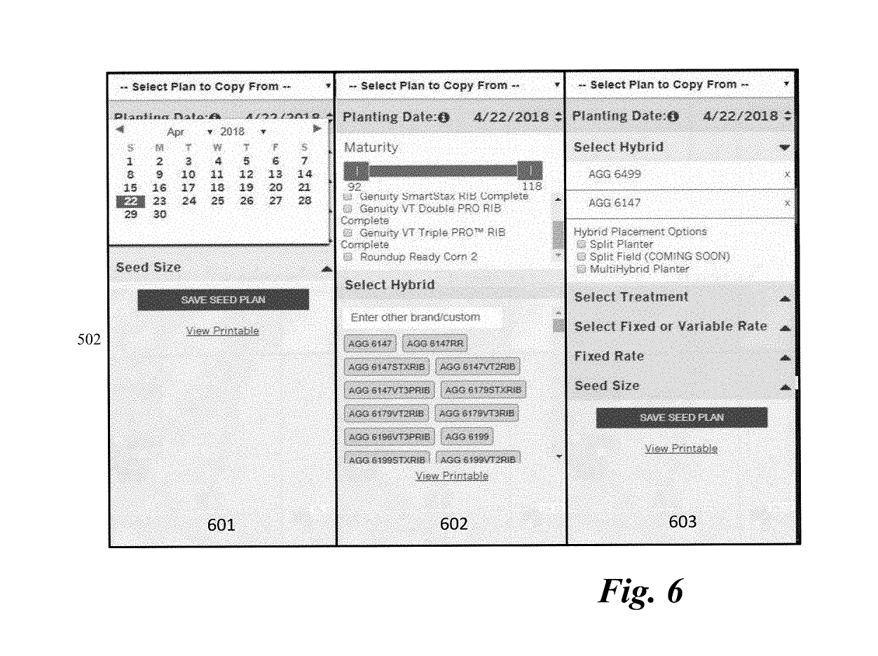

In the illustrative embodiment, the platform (400) embodies multiple planning and recording panes (501) as shown in FIG. 5. For example, a Seed Plan (502) is one of multiple input panes that allows the subscriber to input variables for an assigned geospatial unit regarding planting. The Seed Plan (502) is comprised of several records where additional input variables define the record. As shown in FIG. 6, the record may consist of planting date, hybrid selection, treatment, rate, and seed size. Planting date (601) is manually entered or generated from the platform through optimization algorithms. Hybrid selection (602) consists of selecting a hybrid or hybrids for the assigned geospatial unit, which imbeds specific hybrid information into the record. The hybrids may be optimally placed by the platform (603) through split planting, split field, or multi-hybrid planting.

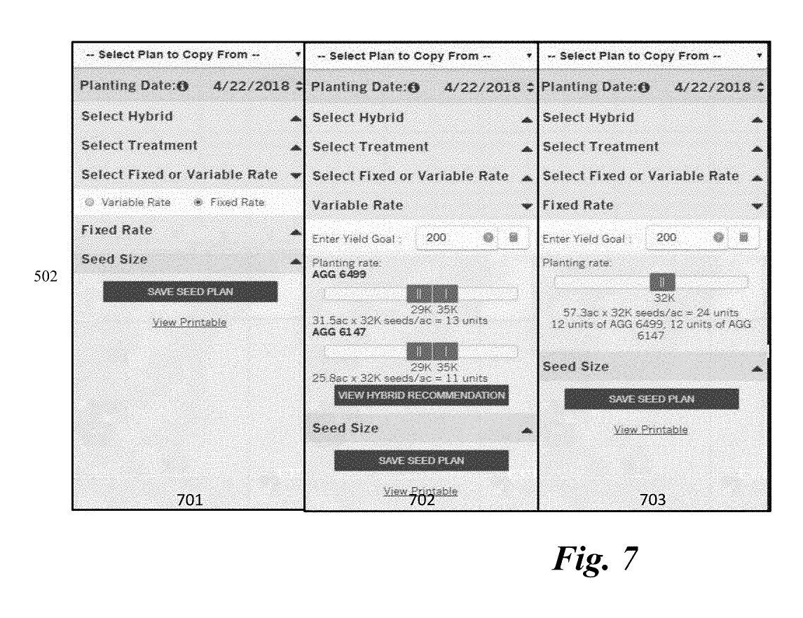

Moreover, as shown in FIG. 7, the Seed Plan (502) further includes several records where additional input variables define the record. The record may include planting date, hybrid selection, treatment, rate, and seed size. Fixed or variable rate planting (701) may be chosen to calculate rates from algorithms based on yield goal as the outcome parameter (702 and 703). Rates may be manually manipulated. Total seed units are calculated for the hybrid(s) (702 and 703).

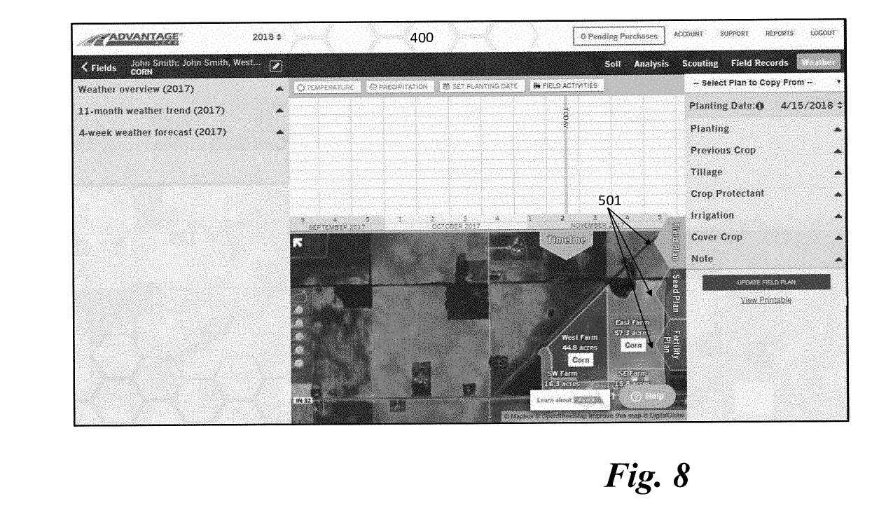

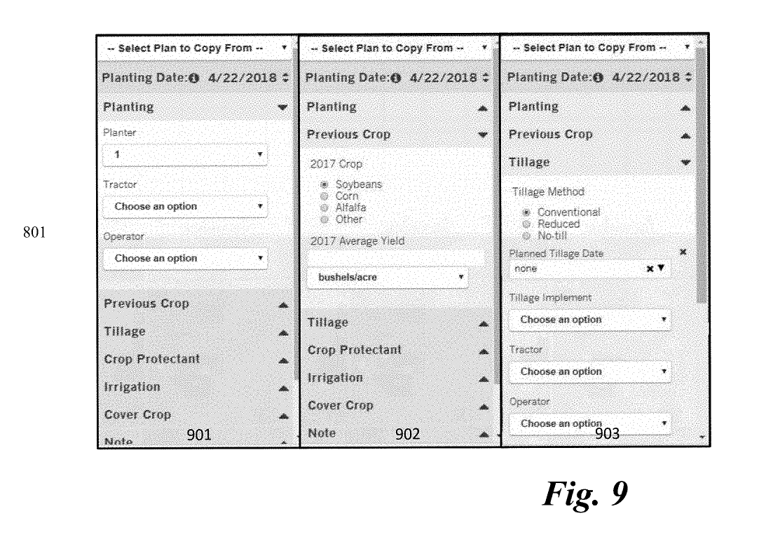

Referring now to FIG. 8, a Field Plan (801) is one of multiple input panes that allows the subscriber to input variables for an assigned geospatial unit. The Field Plan (801) is comprised of several records where additional input variables define the record as shown in FIG. 9. The record may consist of planting date (601), planting, previous crop, tillage, crop protectant, irrigation, cover crop, and notes. Planting (901) includes input of equipment and operator. Previous crop (902) allows for input of previously planted crop and average yield. Tillage (903) allows for input of tillage methods and details, equipment and operator, of the event.

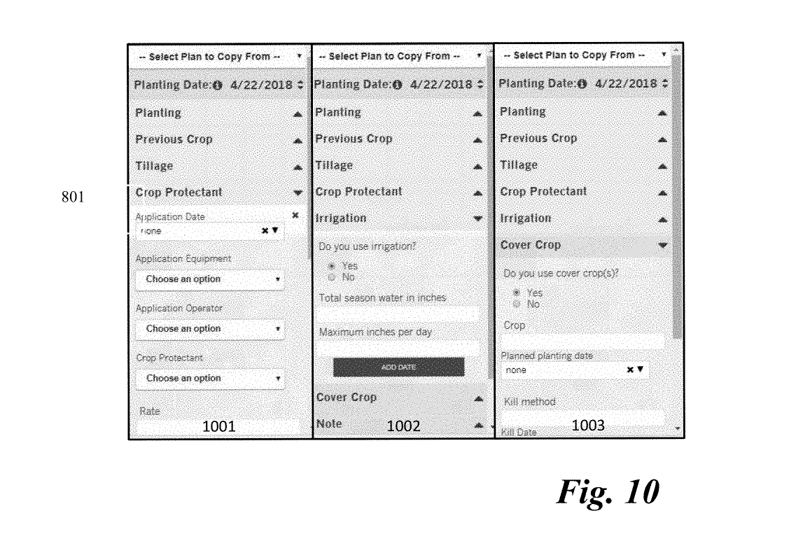

As shown in FIG. 10, the Field Plan (801) is further comprised of several records where additional input variables define the record. The record may consist of planting date (601), planting, previous crop, tillage, crop protectant, irrigation, cover crop, and notes. The crop protectant record (1001) allows the input of application and chemistry details. Irrigation (1002) variables include total water usage for the season and maximum inches of irrigation per day. The cover crop record (1003) includes crop and planting details, along with method and kill date.

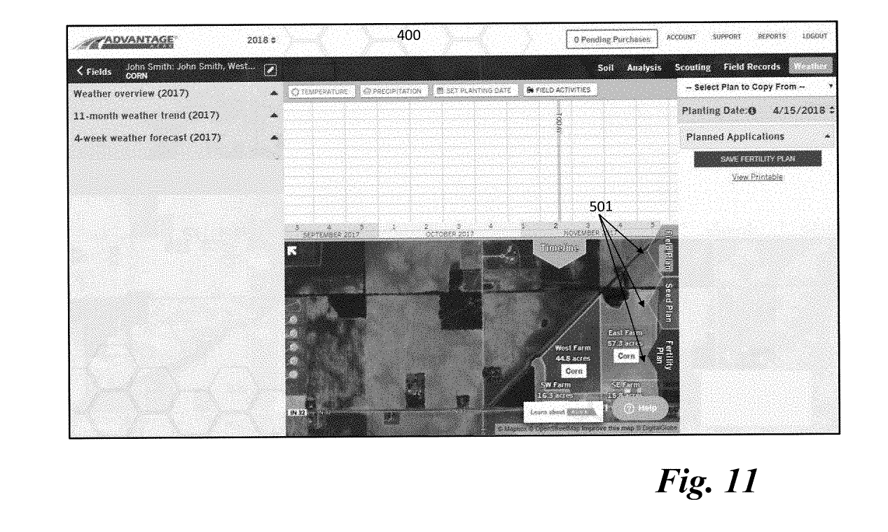

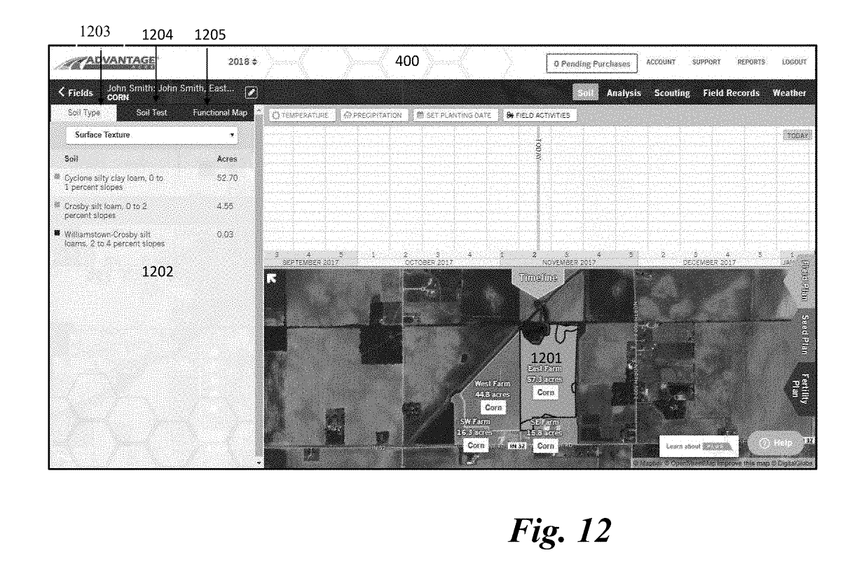

Referring now to FIG. 11, a Fertility Plan (1101) is one of multiple input panes that allows the subscriber to input variables for an assigned geospatial unit. The Fertility Plan allows for the input of fertilizer and manure applications in detail. The platform (400) may allow detailed mapping of soil characteristics for the defined geospatial unit (1201) as shown in FIG. 12. Surface Texture, organic matter %, CEC, pH, drainage, and depth to restrictive soil layer (1202) may be viewed and analyzed (1203). Soil test data can be uploaded and viewed into the platform (1204). A functional soil map may also be generated and analyzed, along with productivity index and functional soil survey (1205).

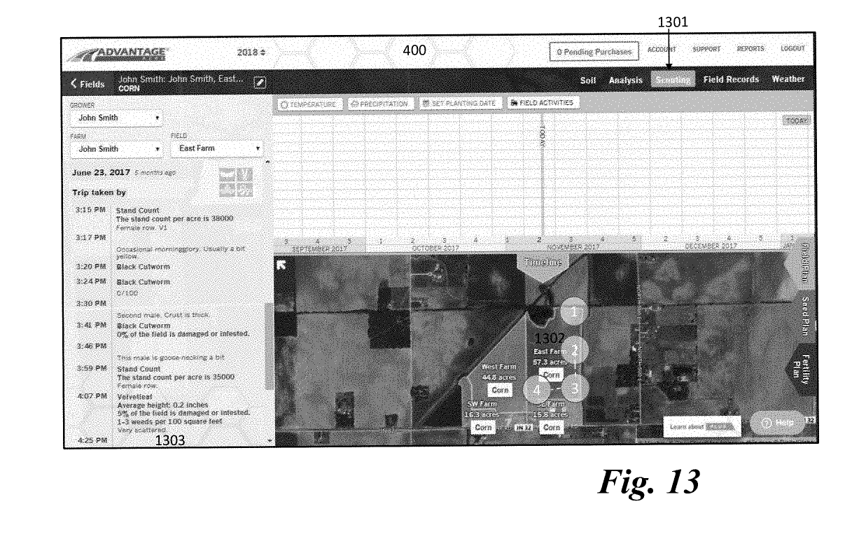

As shown in FIG. 13, the platform (400) may allow recording of detailed scouting information (1301). Scouting trips are recorded and viewable on the map of the geospatial unit through GPS tracking of the user movement and point determination by the user (1302). Information is recorded for each scouting trip by timestamp (1303). A scouting trip records time, notes, photos, and/or information from a selectable pest library.

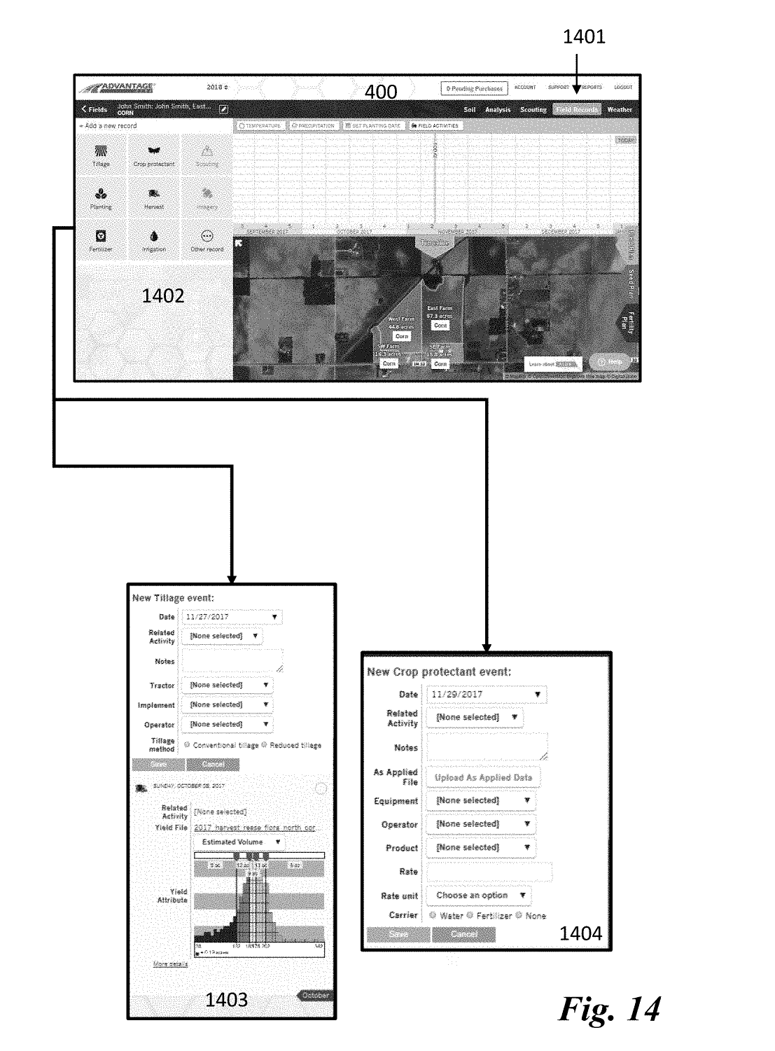

Additionally, the platform (400) may allow for the addition of additional records for a defined geospatial unit (1401) as shown in FIG. 14. Records that may be inputted for the correlating geospatial unit include: tillage, crop protectants, scouting, planting, harvest, imagery, fertilizer, irrigation, and other non-defined management activities (1402). Records can be manually entered or uploaded from as-occurred files from field activities. Additional parameters may be completed for each record (1403 and 1404). Uploaded as-occurred record data may produce an overlay map of the data for the defined geospatial unit and histogram of the bulk data (1403).

Current Disease Risk

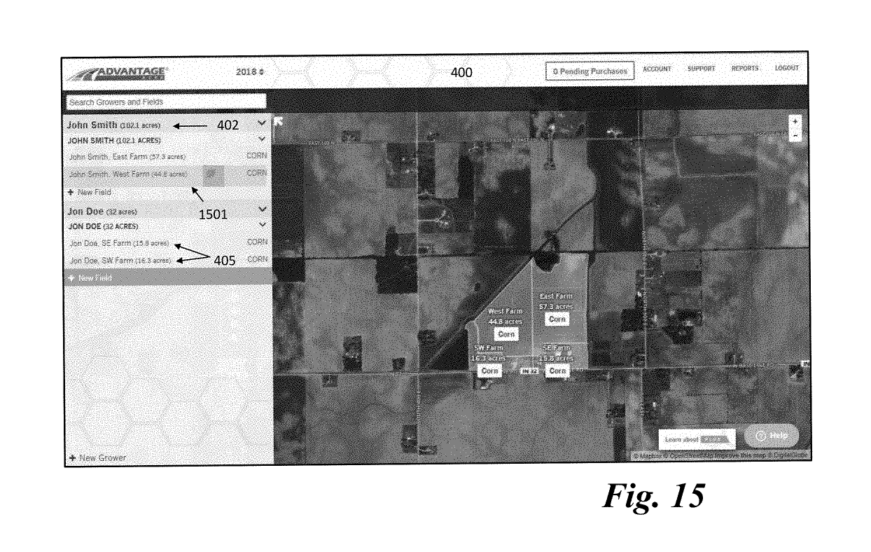

The platform (400) executed on the server computing device may notify a user of a disease risk when models indicate current conditions are conducive to disease. Grower (402) and field (405) lists may be one indicator in the platform for the disease risk. If the models indicate the current conditions are conducive for disease, the effected geospatial unit will be noted (1501) as shown in FIG. 15.

The platform executed on the server computing device is configured to run simulations of a disease risk assessment to determine a current disease risk during crucial plant growth stages, where the disease risk assessment and management practices are critical. During the disease risk assessment simulation, the platform determines characteristics of the plant seeded in a geospatial unit. For example, the platform may determine a hybrid resistance (DR) level of the plant, which may be obtained from a supplier or a manufacturer of the hybrid plant/seed. If the DR level is greater than a first threshold, the plant is considered a high resistance crop and the platform generates a “Low Risk” visual representation to output information associate with the “Low Risk” on the computing device. In the illustrative embodiment, the platform may not further perform a modeled disease risk assessment to determine a disease risk at the geospatial unit if the plant is considered a low risk based on the characteristics of the plant.

If, however, the DR level is within a predefined range (i.e., less the first threshold and greater than a second threshold), the plant is considered a medium resistance crop and the platform further monitors whether environmental variables (e.g., temperature (F), rainfall (RV), and relative humidity (RH)) satisfy a predefined disease condition based on the present and previous weather data. If the environmental variables satisfy the predefined disease condition, the platform may further consider one or more soil variables (e.g., crop residue from previous crop, tillage practices) to determine a current disease risk of the plant growing in the defined geospatial unit.

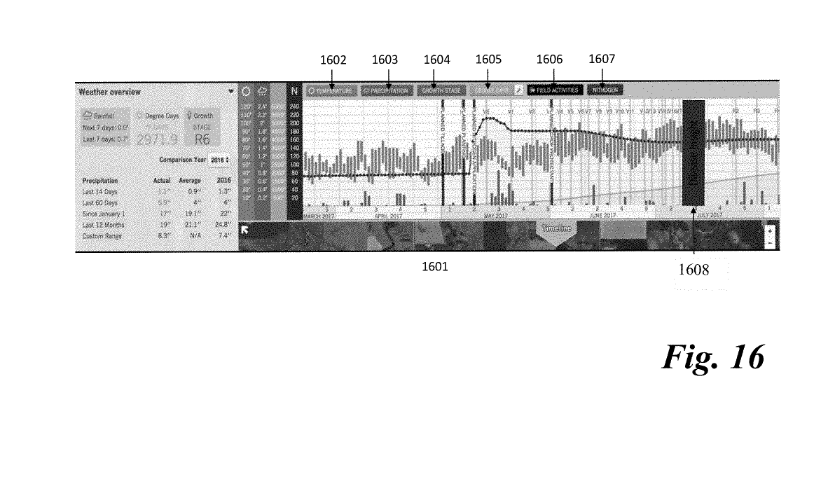

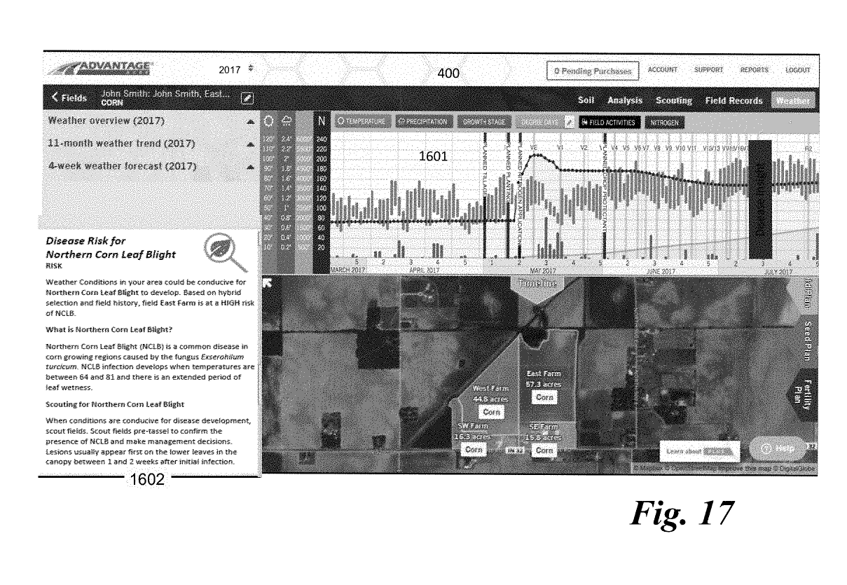

For example, to predict a current disease risk for Gray Leaf Spot, the platform determines the hybrid resistance (DR) level of the plant seeded in a geospatial unit. If the DR level is greater than 7, the plant is considered a high resistance crop and the platform indicates “Low Risk” on the visual representation of the computing device. If, however, the DR level is between 4 and 6, the plant is considered a medium resistance crop. Accordingly, the platform further determines whether the environmental variables satisfy a predefined disease condition of Gray Leaf Spot. Specifically, the platform determines whether (i) the temperature (F) is between 70 and 85° F. and (ii) the relative humidity (RH) is greater than 90% or the chance of precipitation (RVC) is at least 80% based on a daily forecast data. If the environmental variables satisfy a predefined disease condition of Gray Leaf Spot, the platform further determines the crop residue from previous crop and tillage practices of the corresponding geospatial unit to generate a visual representation of the current disease risk as shown in FIGS. 16 and 17.

As illustrated in FIG. 16, the timeline depicts activities and parameters for a defined geospatial unit, which may include a weather summary (1601). Both forecasted and historic data are present on the timeline. Actual temperature highs and lows are represented daily (1602). Actual precipitation is recorded daily (1603). Predicted crop progress is calculated and indicated on the timeline by growth stage (1604). Growing degree days (GDD) are tracked, accumulated, and predicted on the timeline (1605). Field activities that are recorded in field records (1401) are indicated on the date of occurrence on the timeline (1606). Available nitrogen estimates may also be predicted (1607). If the disease models indicate that current conditions are conducive to disease, the risk will appear on the timeline (1608).

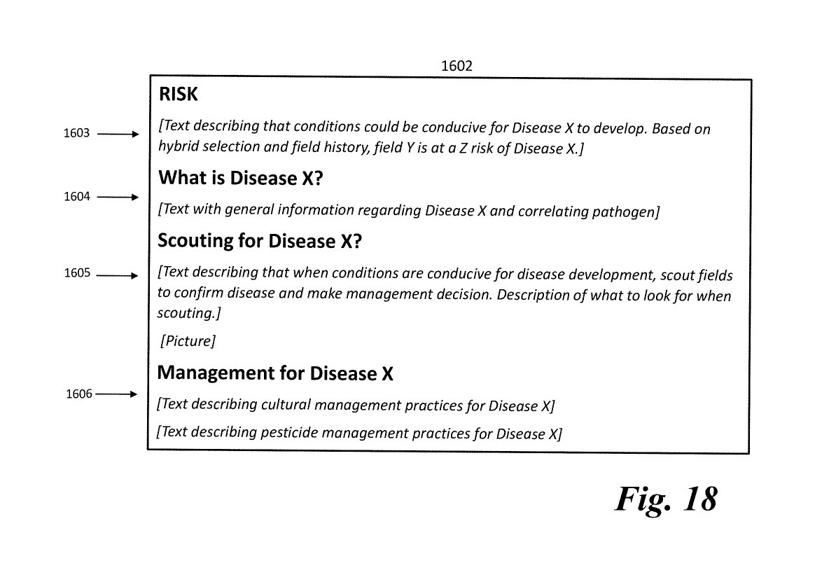

When viewing the defined geospatial unit in the platform (400) as shown in FIG. 17, the timeline may appear with the disease risk indication (1601). A detailed insight into current disease risk may appear with management resources (1602). For example, as shown in FIG. 18, the disease insight (1602) may include: a description of why the disease risk model indicated risk (1603), information regarding the disease and pathogen (1604), pertinent scouting information such as signs, symptoms, and picture of the disease (1605), and/or cultural and chemical management practices for the disease (1606). Output may include (i) a description of a disease that is conducive to develop based on conditions (e.g., based on hybrid selection and field history, field Y is at a Z risk of Disease X), (ii) a description of the disease (e.g., general information regarding Disease X and correlating pathogen), (iii) a description of what to look for when scouting fields to confirm disease and make management decision, (iv) a description of cultural and/or pesticide management practices for the disease.

Short-Term Forecast Disease Risk

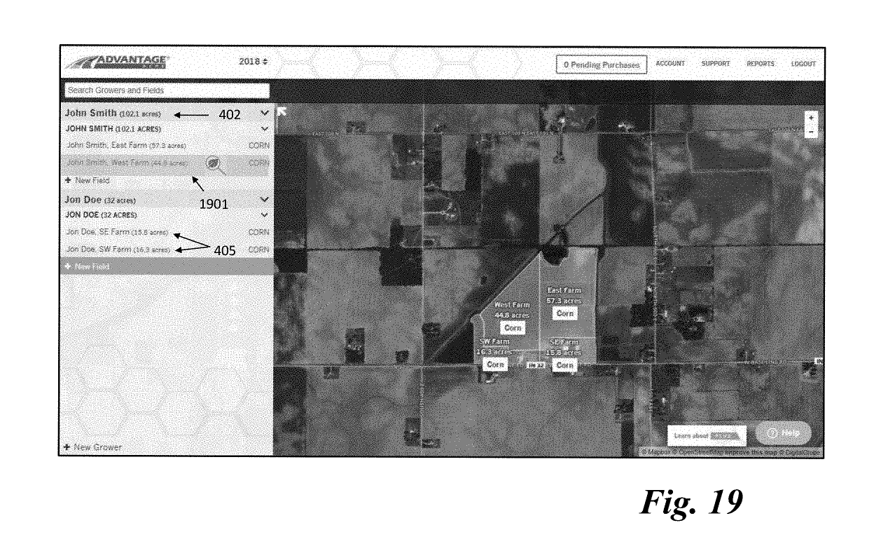

In some embodiments, if the models indicate that short-term forecasts are conducive for disease, the effected geospatial unit will be noted (1901) to notify the user as shown in FIG. 19. As discussed above, Growers (402) and field (405) lists may be one indicator in the platform for disease risk.

The platform executed on the server computing device is configured to run simulations of a disease risk assessment to determine a short-term forecast disease risk during crucial plant growth stages, where disease risk assessment and management practices are critical. During the disease risk assessment simulation, the platform determines characteristics of the plant seeded in a defined geospatial unit defined by a subscriber. For example, the platform may determine a hybrid resistance (DR) level of the plant, which may be obtained from a supplier or a manufacturer of the hybrid plant/seed. If the DR level is greater than a first threshold, the plant is considered a high resistance crop and the platform generates a “Low Risk” visual representation to output information associate with the “Low Risk” on the computing device. In the illustrative embodiment, the platform does not perform a modeled disease risk assessment to determine a disease risk at the geospatial unit if the plant is considered a low risk based on the characteristics of the plant.

If, however, the DR level is within a predefined range (i.e., less the first threshold and greater than a second threshold), the plant is considered a medium resistance crop and the platform further monitors whether environmental variables (e.g., temperature (F), rainfall (RV), and relative humidity (RH)) satisfy a predefined disease condition based on weather forecast data for next predefined period of time. If the future environmental variables satisfy the predefined disease condition, the platform may further consider one or more soil variables (e.g., crop residue from previous crop, tillage practices) to determine a short-term disease risk of the plant growing in the defined geospatial unit.

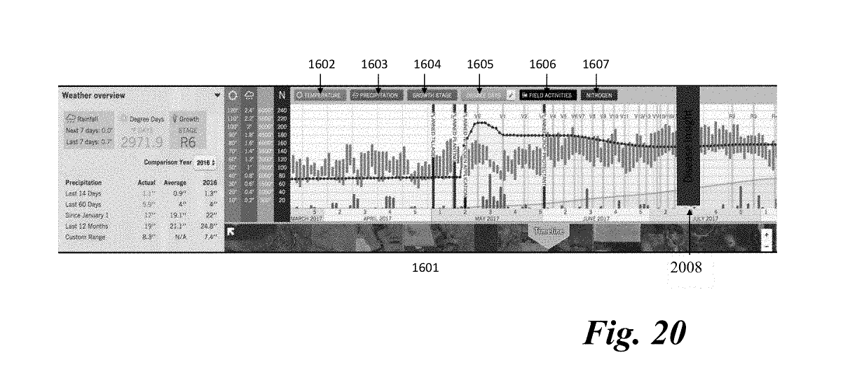

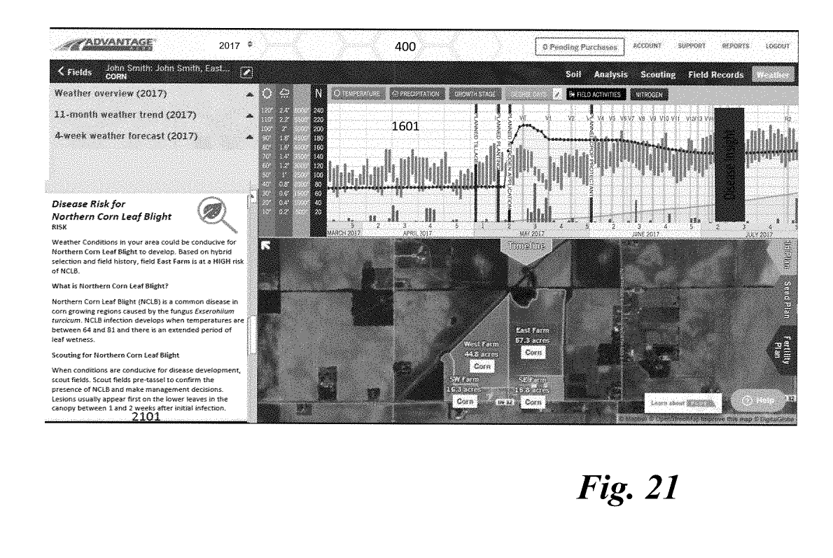

For example, to predict a short-term disease risk for Gray Leaf Spot, the platform determines the hybrid resistance (DR) level of the plant seeded in a defined geospatial unit defined by a subscriber as a high resistance. If the DR level is greater than 7, the plant is considered a high resistance crop and the platform indicates “Low Risk” on the visual representation of the computing device. If, however, the DR level is between 4 and 6, the plant is considered a medium resistance crop. Accordingly, the platform further determines whether the environmental variables satisfy a predefined disease condition of Gray Leaf Spot. Specifically, the platform determines whether (i) the temperature (F) is between 70 and 85° F. and (ii) the relative humidity (RH) is greater than 90% or the chance of precipitation (RVC) is at least 80% based on the weather forecast data for next 7 consecutive days. If the environmental variables satisfy a predefined disease condition of Gray Leaf Spot, the platform further determines the crop residue from previous crop and tillage practices of the corresponding geospatial unit to generate a visual representation of the short-term disease risk as shown in FIGS. 20 and 21. It should be appreciated that the platform of the server computing device may combine a current and short-term disease risks.

As illustrated in FIG. 20, the timeline depicts activities and parameters for a defined geospatial unit, which may include a weather summary (1601). Both forecasted and historic data are present on the timeline. Actual temperature highs and lows are represented daily (1602). Actual precipitation is recorded daily (1603). Predicted crop progress is calculated and indicated on the timeline by growth stage (1604). Growing degree days (GDD) are tracked, accumulated, and predicted on the timeline (1605). Field activities that are recorded in field records (1401) are indicated on the date of occurrence on the timeline (1606). Available nitrogen estimates may also be predicted (1607). If the disease models indicate that short-term forecasted conditions are conducive to disease, the risk will appear on the timeline (2008). When viewing the defined geospatial unit in the platform (400), the timeline may appear with the disease risk indication (1601).

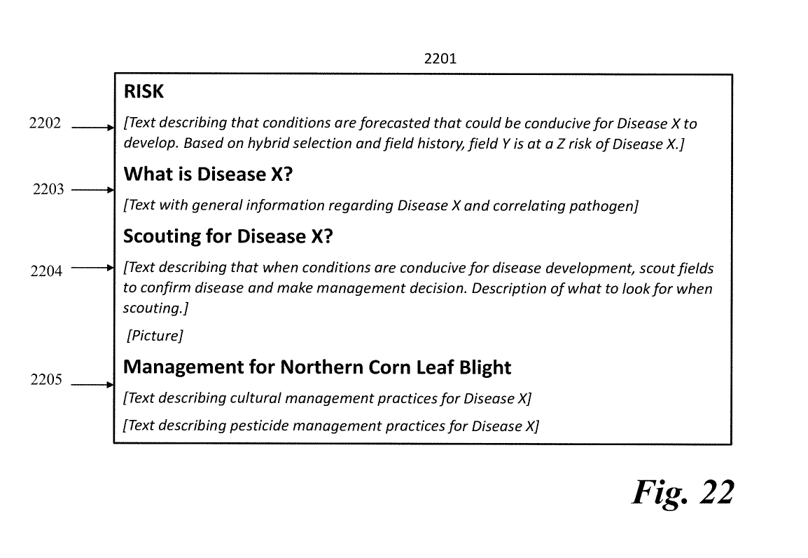

A detailed insight to short-term forecasted disease risk may appear with management resources (2101) as shown in FIG. 21. For example, as shown in FIG. 22, the disease insight (2201) may include: a description of why the disease risk model indicated risk (2202), information regarding the disease and pathogen (2203), pertinent scouting information such as signs, symptoms, and picture of the disease (2204), and/or cultural and chemical management practices for the disease (2205). Output may include (i) a description of a disease that is conducive to develop based on conditions (e.g., based on hybrid selection and field history, field Y is at a Z risk of Disease X), (ii) a description of the disease (e.g., general information regarding Disease X and correlating pathogen), (iii) a description of what to look for when scouting fields to confirm disease and make management decision, (iv) a description of cultural and/or pesticide management practices for the disease.

It should be appreciated that the platform (400) executed on the server computing device periodically performs disease risk assessments for the current risk and the short-term forecast risk and notifies the user the current and short-term disease risks. For example, the platform may generate visual indications on the timeline displayed on the computing device at which the disease risk predictions exceed the corresponding predefined threshold. In other words, the platform may inform the user the times at which the plants in the geospatial unit are likely to develop a disease based on the current and short-term forecast disease risk assessment. Additionally, the platform may further provide the user information regarding a pathogen related to the disease, information regarding the disease, scouting information, general management and cultural management practices, and/or pesticide specific management practices.

Long-Term Forecast Disease Risk

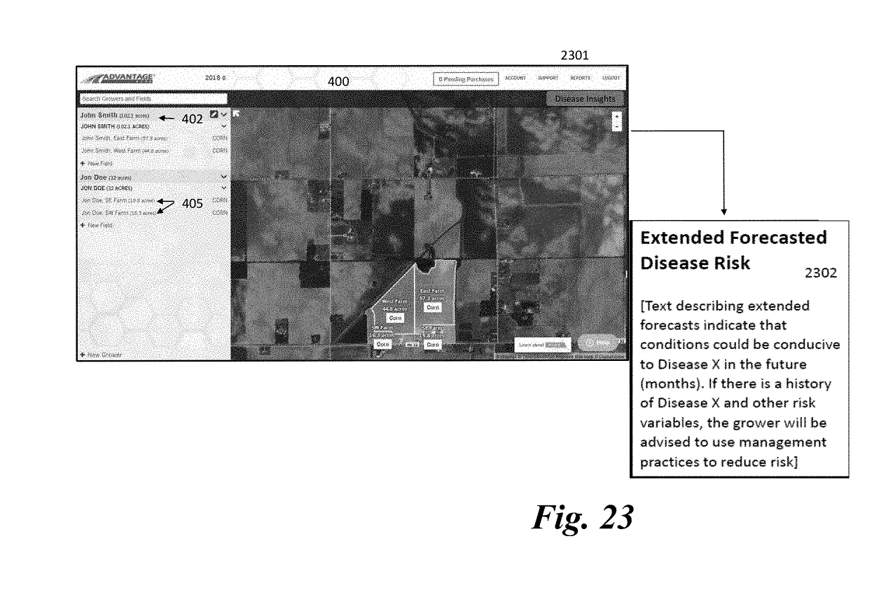

In some embodiments, if the models indicate that long-term forecasts are conducive for disease, a disease insight icon (2301) may appear for further insights into disease for the season to notify the user as shown in FIG. 23. As discussed above, Growers (402) and field (405) lists may be one indicator in the platform for disease risk. Additionally, the long-term disease risk insight (2302) may indicate the conditions that initiated the model risk and management resources.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A computing system to assess a disease risk, the computing system comprising:
a computing device; and
a server computing device communicatively coupled to the computing device, the server computing device configured to:
receive a geographical area of a geospatial unit defined by a user of the computing device;
receive input variables associated with the geospatial unit from one or more input sources of the computing device;
determine a disease risk of plants at the geospatial unit by performing a modeled disease risk assessment as a function of the input variables and a set of predefined variables;
generate a visual representation that illustrates the disease risk based on the modeled disease risk assessment;
output the visual representation on the computing device; and
transmit, in response to determining that the disease risk exceeds a predefined threshold, a notification to a user of the computing device indicating that the geospatial unit is at risk.

2. The computing system of claim 1, wherein to generate the visual representation that illustrates the disease risk at the geospatial unit comprises to generate a visual indication on a timeline at which the disease risk exceeds the predefined threshold, wherein the visual indication represents specific time at which the plants in the geospatial unit are likely to develop a disease.

3. The computing system of claim 1, wherein to determine the disease risk comprises to analyze the input variables to determine current, short-term, and/or long-term disease risk levels for individual plant pathogens in the geospatial unit.

4. The computing system of claim 1, wherein the server computing device is further configured to receive a plurality of geospatial units and simultaneously determine a disease risk at each geospatial unit by performing the modeled disease risk assessment as a function of the input variables and a set of predefined variables for the each geospatial unit.

5. The computing system of claim 1, wherein to determine the disease risk comprises to determine a hybrid resistance level of the plants at the geospatial unit, and to generate the visual representation comprises to change the visual representation based on the hybrid resistance level.

6. The computing system of claim 5, wherein to determine the disease risk comprises to generate a low risk visual representation on the computing device in response to determining that the hybrid resistance level is above a predefined level.

7. The computing system of claim 5, wherein to determine the disease risk comprises to determine whether secondary input variables of the geospatial unit satisfy a predefined disease condition in response to determining that the hybrid resistance level is within a predefined range.

8. The computing system of claim 7, wherein the secondary input variables include temperature, relative humidity, and chance of precipitation.

9. The computing system of claim 7, wherein to determine the disease risk comprises to determine, in response to determining that the secondary input variables satisfy the predefined disease condition, crop rotation history and tillage practices of the geospatial unit to determine the disease risk and determine whether the disease risk exceeds the predefined threshold.

10. The computing system of claim 9, wherein to generate the visual representation on the computing device comprises to generate, in response to determining that the disease risk exceeds the predefined threshold, the visual representation indicative of the disease risk at the geospatial unit that includes information regarding a pathogen related to the disease, information regarding the disease, scouting information, general management and cultural management practices, and/or pesticide specific management practices.

11. A method for assessing a disease risk, the method comprising:
receiving a geographical area of a geospatial unit defined by a user of the computing device;
receiving input variables associated with the geospatial unit from one or more input sources of the computing device;
determining the disease risk of plants at the geospatial unit by performing a modeled disease risk assessment as a function of the input variables and a set of predefined variables;
generating a visual representation that illustrates the disease risk based on the modeled disease risk assessment;
outputting the visual representation on the computing device; and
transmitting, in response to determining that the disease risk exceeds a predefined threshold, a notification to a user of the computing device indicating that the geospatial unit is at risk.

12. The method of claim 11, wherein generating the visual representation that illustrates the disease risk at the geospatial unit comprises generating a visual indication on a timeline at which the disease risk exceeds the predefined threshold, wherein the visual indication represents specific time at which the plants in the geospatial unit are likely to develop a disease.

13. The method of claim 11, wherein determining the disease risk comprises analyzing the input variables to determine current, short-term, and/or long-term disease risk for individual plant pathogens in the geospatial unit.

14. The method of claim 11 further comprising receiving a plurality of geospatial units and simultaneously determine a disease risk at each geospatial unit by performing the modeled disease risk assessment as a function of the input variables and a set of predefined variables for the each geospatial unit.

15. The method of claim 11, wherein determining the disease risk comprises determining a hybrid resistance level of the plants at the geospatial unit, and generating the visual representation comprises changing the visual representation based on the hybrid resistance level.

16. The method of claim 15, wherein determining the disease risk comprises generating a low risk visual representation on the computing device in response to determining that the hybrid resistance level is above a predefined level.

17. The method of claim 15, wherein determining the disease risk comprises determining whether secondary input variables of the geospatial unit satisfy a predefined disease condition in response to determining that the hybrid resistance level is within a predefined range.

18. The method of claim 17, wherein the secondary input variables include temperature, relative humidity, and chance of precipitation.

19. The method of claim 17, wherein determining the disease risk comprises determining, in response to determining that the secondary input variables satisfy the predefined disease condition, crop rotation history and tillage practices of the geospatial unit to determine the disease risk and determine whether the disease risk exceeds the predefined threshold.

20. The method of claim 19, wherein generating the visual representation on the computing device comprises generating, in response to determining that the disease risk exceeds the predefined threshold, the visual representation indicative of the disease risk at the geospatial unit that includes information regarding a pathogen related to the disease, information regarding the disease, scouting information, general management and cultural management practices, and/or pesticide specific management practices.
